(19) United States
(12) Patent Application Publication
(10) Pub. No.: US 2025/0228877 A1
(43) Pub. Date: Jul. 17, 2025

(54) MANAGEMENT OF SKIN AILMENTS

(71) Applicant: AbulKalam Shamsuddin, Lutherville, MD (US)

(72) Inventor: AbulKalam Shamsuddin, Lutherville, MD (US)

(21) Appl. No.: 18/705,408

(22) PCT Filed: Oct. 28, 2022
(86) PCT No.: PCT/US2022/078942

§ 371 Date: Apr. 26, 2024

Related U.S. Application Data
(60) Provisional application No. 63/273,787, filed on Oct. 29, 2021.

(51) Int. Cl. A61K 31/6615; A61K 8/19; A61K 8/34; A61K 8/55; A61K 31/05; A61P 17/00; A61P 17/02; A61P 17/06; A61P 35/00; A61Q 19/00; A61Q 19/08

(52) U.S. Cl. CPC A61K 31/6615; A61K 8/19; A61K 8/347; A61K 8/55; A61K 31/05; A61P 17/00; A61P 17/02; A61P 17/06; A61P 35/00; A61Q 19/007; A61Q 19/08

(57) ABSTRACT

The present invention relates to methods and compositions for treating mammalian cells and tissues for erythema, superficial (1^{st} degree) thermal burns, eczema, psoriasis, and other skin ailments including skin rashes, poison ivy, melanoma, carcinoma including basal cell carcinoma, actinic keratosis, ageing-related damages, sun exposure-related damage, red or irritated skin, leukoderma, melasma, shingles, appearance of wrinkles. Compositions of the invention include inositol hexakisphosphate (IP-6) with or without inositol, and pharmaceutically acceptable salts thereof including Ca—Mg-IP6, for topical administration.

### RELATED APPLICATION

This application claims the benefit of U.S. Patent Application No. 63/273,787, filed Oct. 29, 2021, which is incorporated herein by reference in its entirety to the full extent permitted by law.

### TECHNICAL FIELD

The present invention relates to methods and compositions for treating mammalian cells and tissues for erythema, superficial (1st degree) thermal burns, eczema, psoriasis, and other skin ailments such as skin rashes, poison ivy, melanoma, carcinoma including basal cell carcinoma, actinic keratosis, ageing-related damages, sun exposure-related damage, red or irritated skin, leukoderma, melasma, shingles, appearance of wrinkles, etc. In particular, the present invention relates to the use of inositol hexakisphosphate (IP-6) and its derivatives, including pyrophosphate and citrate derivatives, with or without inositol, topically administered to a mammalian subject for treatment of thermal burns, eczema, psoriasis, shingles, and melanoma.

### SUMMARY

Though important physiological and pharmacological functions of IP-6 and inositol in the mammalian body have been demonstrated after being absorbed into the blood stream, there is little to no information on the topical use of IP-6 including with inositol or its safety and efficacy for treating diseases in humans.

One aim of the present disclosure is to provide compositions and methods for treatment of skin ailments such as erythema, rashes, eczema, psoriasis, melanoma, thermal burn, melasma, shingles, leukoderma, basal cell carcinoma, or melanoma. In one embodiment, this invention pertains to topical application of a skin cream composition comprising Ca—Mg-IP-6 and inositol mixed in an approximate 1:1 molar ratio. In another embodiment, the composition is administered topically.

Another aim of the present disclosure is to provide a method for preventing or treating erythema of skin, skin rashes, superficial thermal burn, eczema, psoriasis, non-specific dermatitis, and other skin ailments including melanoma, basal cell carcinoma, leukoderma, shingles, and melasma, in a mammal in need thereof, wherein the method comprises topically applying a therapeutically effective amount of a composition comprising IP-6, its pharmaceutically acceptable salts, or its pharmaceutically acceptable derivatives, in any combination, in an amount from about 0.1% to about 10% by weight of the composition.

Yet another aim of the present disclosure is to provide a method for preventing or treating erythema of skin, skin rashes, superficial thermal burn, eczema, psoriasis, non-specific dermatitis, and other skin ailments including melanoma, basal cell carcinoma, leukoderma, shingles, and melasma, in a mammal in need thereof, wherein the method comprises topically applying a therapeutically effective amount of a composition comprising: (i) IP-6, its pharmaceutically acceptable salts, or its pharmaceutically acceptable derivatives, in any combination, in an amount from about 0.01% to about 10% by weight; and (ii) inositol, its pharmaceutically acceptable salts, or its pharmaceutically acceptable derivatives, in any combination, in an amount from about 0.01% to about 10% by weight.

Another aim of the present disclosure is to provide a composition for preventing or treating erythema of skin, skin rashes, superficial thermal burn, eczema, psoriasis, non-specific dermatitis, and other skin ailments including melanoma, basal cell carcinoma, leukoderma, shingles, and melasma, in a mammal in need thereof, wherein the composition comprises: (i) IP-6, its pharmaceutically acceptable salts, or its pharmaceutically acceptable derivatives, in any combination, in an amount from about 0.01% to about 10% by weight; and (ii) inositol, its pharmaceutically acceptable salts, or its pharmaceutically acceptable derivatives, in any combination, in an amount from about 0.01% to about 10% by weight.

A further aim of the present disclosure is to provide a method for preventing or treating thermal burns of an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

One aim of the present disclosure is to provide a composition used for preventing or treating thermal burns of an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

Another aim of the present disclosure is to provide a method for preventing or treating eczema in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a composition used for preventing or treating eczema in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a method for preventing or treating psoriasis in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

Another aim of the present disclosure is to provide a composition used for preventing or treating psoriasis in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

Yet another aim of the present disclosure is to provide a method for preventing or treating melanoma in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

One aim of the present disclosure is to provide a composition used for preventing or treating melanoma in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

Yet another aim of the present disclosure is to provide a method for preventing or treating basal cell carcinoma in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

One aim of the present disclosure is to provide a composition used for preventing or treating basal cell carcinoma in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a method for preventing or treating erythema in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

Another aim of the present disclosure is to provide a composition used for preventing or treating erythema in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a method for preventing or treating rash in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a composition used for preventing or treating rash in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a method for preventing or treating shingles in an individual in need thereof, wherein the method comprises topically applying a therapeutically effective amount of a composition comprising: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

A further aim of the present disclosure is to provide a composition used for preventing or treating rash in an individual in need thereof, wherein the composition comprises: 4% by weight of Ca—Mg-IP-6, its pharmaceutically acceptable salts, or its pharmaceutically acceptable derivatives, in any combination, and inositol, its pharmaceutically acceptable salts, or its pharmaceutically acceptable derivatives, in any combination.

### BRIEF DESCRIPTION OF THE FIGURES

FIG. 1 depicts the results of Example 8, showing the results of successfully treating the area under the right eye of a patient which had been diagnosed as actinic keratosis with a composition of the invention.

### DETAILED DESCRIPTION OF SPECIFIC EMBODIMENTS

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

### I. INTRODUCTION

Applicant has found, inter alia, that topical compositions comprising IP-6 and inositol are surprisingly effective in treating thermal burns, eczema, psoriasis, basal cell carcinoma, and melanoma and the like. This treatment provides superior results over existing therapies for these conditions.

### II. DEFINITIONS

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values is stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

The term “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable emollients.

The term “inositol” herein means myo-inositol.

Inositol can exist, for example in a phosphate form such as various phosphorylated forms (inositol phosphates or “IPs”), including IP-7 and IP-8. IPs can exist in salt forms as inositol phosphate salts, such as calcium, magnesium, sodium, potassium, or mixtures thereof. A preferred salt form is the calcium magnesium salt. Inositol can also exist in a lipid form such as phosphatidylinositol (PI) and phosphatidylinositol phosphate (PIP) lipids.

In one embodiment, inositol as used herein encompasses pharmaceutically acceptable derivatives of myo-inositol, including esters, salts, or prodrugs of myo-inositol, in any efficacious combination.

The term “IP-6” or the term “InsP₆,” “IP6,” (Scheme I) as used herein refers to myo-inositol hexakisphosphate. In one embodiment, the term IP-6 includes pharmacologically acceptable derivatives of IP-6, including esters, particularly the pyrophosphate and citrate esters of IP-6, for example IP-6 hexacitrate, etc. (IPc), prodrugs of the same, and pharmacologically acceptable salts thereof, in any efficacious combination.

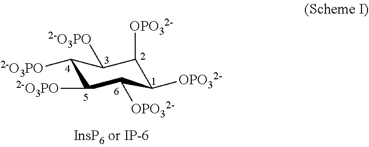

In one embodiment, IP-6 can exist in salt forms such as calcium, magnesium, sodium, potassium, or mixtures thereof. A preferred salt form of IP-6 is the calcium magnesium salt, Ca—Mg-IP-6.

In one embodiment, IP6 Citrate (IPc) is used. Addition of citrate to IP6 molecule gives rise to a new molecule called inositol hexaphosphate citrate (IP6c). While the parent IP6 molecule has 12 valences, the new molecule has 24, making it a better chelator and potentially an even better antioxidant.

The term “derivative” as used herein means any salts, esters (including phosphate, pyrophosphate, phosphocitrate, or citrate esters), or metabolites of the compounds of the invention, including inositol or IP-6.

The terms “subject” or “patient” are used interchangeably herein and refer to a vertebrate, preferably a mammal. Mammals include, but are not limited to, humans.

The term “pharmaceutically-acceptable organic solvent” refers to an organic solvent which, in addition to being capable of having dispersed or dissolved therein the inositol or IP-6 compound, and optionally also an anti-inflammatory or other agent, also possesses acceptable safety (e.g., irritation and sensitization characteristics).

The term “cosmetically-acceptable organic solvent” refers to an organic solvent which, in addition to being capable of having dispersed or dissolved therein the inositol or IP-6 compound, and optionally also an anti-inflammatory or other agent, also possesses good aesthetic properties (e.g., does not feel greasy or tacky).

The terms “therapeutically effective” or “therapeutically-effective amount,” as used herein, refer to the amount of the biologically active agent needed to stimulate or initiate the desired beneficial result. The amount of the biologically active agent in the pharmaceutical compositions of the invention will be that amount necessary to deliver an amount of the biologically active agent needed to achieve the desired result. In practice, this will vary widely depending upon the particular biologically active agent being delivered, the site of delivery, and the dissolution and release kinetics for delivery of the biologically active agent into skin tissues.

### II. THE COMPOSITION

In one embodiment, the composition comprises IP-6 and inositol, or pharmaceutically acceptable salts, or derivatives thereof. In another embodiment, the composition comprises pyrophosphate and/or citrate derivatives of IP-6. In yet another embodiment, the composition comprises IP-6 and inositol, pharmaceutically acceptable salts, or derivatives thereof and at least one pharmaceutically acceptable excipient or carrier.

In another embodiment, the composition comprises IP-6, its pharmaceutically acceptable salts, or its pharmaceutically acceptable derivatives, in any combination, in an amount from about 0.1% to about 20%, from about 0.1% to about 19%, from about 0.1% to about 18%, from about 0.1% to about 17%, from about 0.1% to about 16%, from about 0.1% to about 15%, from about 0.1% to about 14%, from about 0.1% to about 13%, from about 0.1% to about 12%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3.2%, from about 0.1% to about 3%, from about 0.1% to about 2%, from about 0.1% to about 1%, from about 1% to about 20%, from about 5% to about 20%, from about 10% to about 20%, from about 15% to about 20%, from about 1% to about 15%, from about 3% to about 10%, from about 1% to about 5%, or from about 5% to about 7.5% by weight of the composition.

In yet another embodiment, the composition comprises IP-6 and inositol, pharmaceutically acceptable salts, or derivatives thereof, in any combination, wherein IP-6, its pharmaceutically acceptable salts, or its pharmaceutically acceptable derivatives is present in an amount from about 0.1% to about 20%, from about 0.1% to about 19%, from about 0.1% to about 18%, from about 0.1% to about 17%, from about 0.1% to about 16%, from about 0.1% to about 15%, from about 0.1% to about 14%, from about 0.1% to about 13%, from about 0.1% to about 12%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 1% to about 20%, from about 1% to about 10%, from about 5% to about 20%, from about 10% to about 20%, from about 15% to about 20%, from about 1% to about 15%, from about 3% to about 10%, from about 1% to about 5%, or from about 5% to about 7.5% by weight of the composition; and wherein inositol, its pharmaceutically acceptable salts, or its pharmaceutically acceptable derivatives is present in an amount from about 0.1% to about 20%, from about 0.1% to about 19%, from about 0.1% to about 18%, from about 0.1% to about 17%, from about 0.1% to about 16%, from about 0.1% to about 15%, from about 0.1% to about 14%, from about 0.1% to about 13%, from about 0.1% to about 12%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 1% to about 20%, from about 1% to about 10%, from about 5% to about 20%, from about 10% to about 20%, from about 15% to about 20%, from about 1% to about 15%, from about 3% to about 10%, from about 1% to about 5%, or from about 5% to about 7.5% by weight of the composition.

In one embodiment, IP6 and inositol are present in an amount totaling about 4% by weight of the composition. In another embodiment, IP6 and inositol are present in an amount totaling from about 1% to about 5% by weight of the composition.

In another embodiment, IP6 is present in about 0.1-5% by weight of the composition and inositol is present in about 0.1-5% by weight of the composition. In another embodiment, IP6 is present in about 1-5% by weight of the composition and inositol is present in about 0.1-2% by weight of the composition. In another embodiment, IP6 is present in about 3-4% by weight of the composition and inositol is present in about 0.5-1% by weight of the composition. In another embodiment, IP6 is present in an amount totaling about 3% by weight of the composition and inositol is present in an amount totaling about 1% by weight of the composition. In another embodiment, IP6 is present in an amount totaling about 3.2% by weight of the composition and inositol is present in an amount totaling about 0.8% by weight of the composition.

In a preferred embodiment, the composition comprises inositol and IP-6, their pharmaceutically acceptable salts, or their pharmaceutically acceptable derivatives, in any combination, in a molar ratio of about 20:1, about 18:1, about 16:1, about 14:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:12, about 1:14, about 1:16, about 1:18 or about 1:20.

In one embodiment, inositol and/or IP-6 are present as the Mg, Ca, Na, K salt or a mixture thereof.

In one particularly preferred embodiment the invention provides a composition comprising Ca—Mg-IP6+inositol at (1:1) molar ratio mixed in a skin cream for topical application.

In one embodiment the invention provides a composition comprising IP-6 and inositol, their pharmaceutically acceptable salts, or their pharmaceutically acceptable derivatives, in any combination, in a molar ratio from about 10:1 to about 1:10, about 4:1, or about 1:1. In yet another embodiment, the molar ratio of IP-6 to inositol in the composition is from about 4:1 to about 1:1. In a further embodiment, the molar ratio of IP-6 to inositol in the composition is about 1:1. In another embodiment, the molar ratio of IP-6 to inositol in the composition is about 4:1. In other embodiments, the ratio of IP-6 to inositol in the composition is from about 10:1 to about 1:10, about 4:1, or about 1:1 w/v. In one embodiment, the composition comprises 4% by weight of a mixture of IP-6 and inositol.

In another embodiment, the concentration of inositol, its pharmaceutically acceptable salts, or its pharmaceutically acceptable derivatives in the composition is up to about 4% by weight.

In yet another embodiment, the concentration of IP-6, its pharmaceutically acceptable salts, or its pharmaceutically acceptable derivatives in the composition is up to about 4% by weight. In another embodiment, inositol is present in about 3.2% and IP-6 is present in about 0.88% by weight.

In one embodiment, about 1 mg to 1 g of inositol and about 1 mg to 1 g of IP-6 are present in the composition. In another embodiment, about 100 mg of inositol and about 400 mg of IP-6 are present in the composition. In yet another embodiment, about 110 mg of inositol and about 400 mg of IP-6 are present in the composition. In a further embodiment, about 880 mg of inositol and 3.2 g of IP-6 are present in 100 g of the composition.

In another embodiment, the composition of the invention is a cream.

In yet another embodiment, the composition is administered daily. In another embodiment, the composition is administered at least once, at least twice, at least three times, or at least four times daily, or more. In another embodiment, the composition is administered weekly.

In one embodiment, the composition is administered in at least one dose containing a total of about 1 gram to about 10 grams of inositol, IP-6, their pharmaceutically acceptable salts, or their pharmaceutically acceptable derivatives, in any combination. In another embodiment, the composition is administered in at least one dose containing about 0.5 g to 2 grams of inositol and IP-6. In another embodiment, the composition is administered in at least one dose containing about 2 grams to about 5 grams of IP-6. In another embodiment, the composition is administered in at least one dose containing about 1 gram of inositol and/or IP-6.

The active ingredient may be administered at once or may be divided into a number of smaller doses to be administered simultaneously or at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular Subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The specific dose and schedule of inositol and IP-6 composition administration to obtain the desired benefit will, of course, be determined by the particular circumstances of the individual patient including, the size, weight, age and sex of the patient, the nature and stage of the disease being treated, the aggressiveness of the disease, and the route of administration. For example, a daily dosage of from about 0.01 to about 150 mg/kg/day may be utilized, more preferably from about 0.05 to about 50 mg/kg/day. Particularly preferred are doses from about 1.0 to about 40.0 mg/kg/day, for example, a dose of about 30 mg/kg/day. The dose may be given over multiple administrations, for example, two administrations of 15 mg/kg. Higher or lower doses are also contemplated.

In one embodiment, the composition further comprises an antioxidant, a biocide, a chemotherapeutic, a nutritional supplement or nutraceutical, an analgesic, a sunblock, a moisturizer, or any combination thereof.

In another embodiment, the composition is applied in a dosage form comprising inositol, IP-6, their pharmaceutically acceptable salts, or their pharmaceutically acceptable derivatives, in any combination in an amount of about 0.1 g, about 0.5 g, about 1.5 g, about 2 g, about 0.25 g, about 3 g, about 3.5 g, about 4 g, about 4.5 g, or about 5 g each.

In yet another embodiment, the composition is provided in the form of a liquid, lotion, cream, gel or an ointment.

In an embodiment, the IP-6 and/or inositol compounds and compositions of the invention protect and support healthy cell growth. In another embodiment, the IP-6 and/or inositol compounds and compositions of the invention protect normal cells and tissues from the effects of inflammation. In another embodiment, the IP-6 and/or inositol compounds and compositions of the invention provide immune support to cells and tissues. In another embodiment, the inositol and/or IP-6 compounds and compositions of the invention protect normal cells and tissues from the effects of acute and/or chronic exposure to radiation, for example ionizing radiation, solar radiation, etc.

### Topical Compositions

Topical products occur in a variety of forms, including solids, liquids, suspensions, semisolids (such as creams, gels, pastes or “sticks”), powders or finely dispersed liquids such as sprays or mists.

Examples of topical products include compositions commonly classified as “cosmetics”. Such cosmetics include skin care products such as creams, lotions, moisturizers and “treatment cosmetics” such as exfoliants and/or skin cell renewal agents; fragrances such as perfumes and colognes, and deodorants; shaving-related products such as creams, “bracers” and aftershaves; depilatories and other hair removal products; skin cleansers, toners and astringents; pre-moistened wipes and washcloths; tanning lotions; bath products such as oils; eye care products such as eye lotions and makeup removers; foot care products such as powders and sprays; skin colorant and make-up products such as foundations, blushes, rouges, eye shadows and liners, lip colors and mascaras; lip balms and sticks; hair care and treatment products such as shampoos, conditioners, colorants, dyes, bleaches, straighteners and permanent wave products; baby products such as baby lotions, oils, shampoos, powders and wet wipes; feminine hygiene products such as deodorants and douches; skin or facial peels applied by dermatologists or cosmeticians; and others.

Other topical products include hand, facial and body soaps and detergents and other forms of skin cleansers, as well as household detergents and many other household products such as solvents, propellants, polishes, lubricants, adhesives, waxes and others which are either applied topically or are topically exposed to the body during normal use.

Cosmetic compositions are usually inconceivable without the customary auxiliaries and additives such as consistency-imparting agents, fillers, perfume, colorants, emulsifiers, additional active compounds such as vitamins or proteins, sunscreens, stabilizers, insect repellents, alcohol, water, salts, and substances having antimicrobial, proteolytic or keratolytic activity.

“Sunblock” as used herein refers to an active ingredient in sunscreen. Sunscreen can be referred to by many names, including sunblock, sun cream, suntan lotion, and sunblocker. Sunscreens comprise photoprotective active ingredients that are applied topically to the skin to absorb or reflect the sun's damaging ultraviolet (UV) rays. Active ingredients in sunscreens are useful in combination with the invention and include but are not limited to: titanium dioxide, zinc oxide, oxybenzone, avobenzone, homosalate, octinoxate, octocrylene, and octyl salicylate.

Suitable topical vehicles for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol and dimethicone copolyol (Dow Corning); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art.

The topical vehicle may further comprise components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The Extra Pharmacopoeia (Pharmaceutical Press, London 1993) and Martin (ed.), Remington's Pharmaceutical Sciences.

The choice of a suitable vehicle depends on the particular physical form and mode of formulation delivery. Examples of suitable forms include liquids (including dissolved forms of the IP-6 and/or inositol compositions of the invention as well as suspensions, emulsions and the like); solids and semisolids such as gels, foams, pastes, creams, ointments, “sticks” (as in lipsticks or underarm deodorant sticks), powders and the like; formulations containing liposomes or other delivery vesicles; rectal or vaginal suppositories, creams, foams, gels, ointments, enemas or douches; and other forms.

In a specific embodiment, the composition of the invention is a cream.

In one embodiment, the composition of the invention comprises one or more excipients. In another embodiment, the excipient can be colloidal oatmeal, water, glyceryl stearate, cetearyl olivate, cetyl alcohol, ethylhexylglycerin, glycerin, Helianthus annuus oil, phenoxyethanol, sorbitan olivate, xanthan gum or zenea propanediol, or any combination thereof.

In another embodiment, an active ingredient in the composition can be colloidal oatmeal. Excipients can be water, glyceryl stearate, cetearyl olivate, cetyl alcohol, ethylhexylglycerin, glycerin, Helianthus annuus oil, phenoxyethanol, sorbitan olivate, xanthan gum or zenea propanediol, or any combination thereof.

In a specific embodiment, the composition of the invention is a cream containing 1% colloidal oatmeal, water (e.g., deionized water), glyceryl stearate, cetearyl olivate, cetyl alcohol, ethylhexylglycerin, glycerin, Helianthus annuus oil, phenoxyethanol, sorbitan olivate, xanthan gum and zenea propanediol.

In another specific embodiment, the composition of the invention is a cream containing 1% colloidal oatmeal, Ca—Mg-IP-6, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol.

In yet another specific embodiment, the composition of the invention is a cream containing 1% colloidal oatmeal, inositol, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol.

In yet another specific embodiment, the composition of the invention is a cream containing 1% colloidal oatmeal, Ca—Mg-IP-6, inositol, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol.

Typical modes of delivery include application using the fingers; application using a physical applicator such as a cloth, tissue, swab, stick or brush (as achieved for example by soaking the applicator with the formulation just prior to application, or by applying or adhering a prepared applicator already containing the formulation—such as a treated or pre-moistened bandage, wipe, washcloth or stick—to the skin); spraying (including mist, aerosol or foam spraying); dropper application (as for example with ear or eye drops); sprinkling (as with a suitable powder form of the formulation); soaking; and injection (particularly intradermal or subcutaneous injection).

Iontophoresis or other electromagnetic-enhanced delivery systems may also be usefully employed, as for example to increase delivery to the dermis.

Methodologies and materials for preparing formulations in a variety of forms are also described in Anthony L. L. Hunting (ed.), “A Formulary of Cosmetic Preparations (Vol. 2)—Creams, Lotions and Milks,” Nacelle Press (England, N.J. 1993); the contents of which are incorporated herein by reference. For example, Chapter 7, pp. 5-14 (oils and gels); Chapter 8, pp. 15-98 (bases and emulsions); Chapter 9, pp. 101-120 (“all-purpose products”); Chapter 10, pp. 121-184 (cleansing masks, creams, lotions); Chapter 11, pp. 185-208 (foundation, vanishing and day creams); Chapter 12, pp. 209-254 (emollients); Chapter 13, pp. 297-324 (facial treatment products); Chapter 14, pp. 325-380 (hand products); Chapter 15, pp. 381-460 (body and skin creams and lotions); and Chapter 16, pp. 461-484 (baby products) provide helpful guidance.

In one embodiment, compositions of the invention are useful for cosmetic uses. In another embodiment, compositions of the invention are useful for medicinal uses.

In one embodiment, the active compounds used according to the invention are included in compositions that comprise other active pharmaceutical ingredients.

Cosmetic compositions within the meaning of the present invention can, for example, be used, depending on their composition, as skin protection cream, cleansing milk, sunscreen lotion, suntan lotion, nourishing cream, day or night cream, etc. It is optionally possible and advantageous to use the compositions according to the invention as a base for pharmaceutical formulations.

It is advantageous to add to the compositions within the meaning of the present invention further anti-irritants or anti-inflammatory active compounds. In one embodiment, the active compound is batyl alcohol (α-octadecylether of glycerol), selachyl alcohol (α-Monooleyl glyceryl ether), chimyl alcohol (3-(hexadecyloxy)-1,2-propanediol), bisabolol and/or panthenol.

It is also advantageous to add to the compositions within the meaning of the present invention customary antioxidants. According to the invention, convenient antioxidants which can be used are all antioxidants suitable or utilizable for cosmetic and/or dermatological applications.

In one embodiment, the antioxidants are selected from the group consisting of amino acids (e.g. glycine, histidine, tyrosine, tryptophan) and their derivatives, imidazoles (e.g. urocanic acid) and its derivatives, peptides such as D,L-carnosine, D-carnosine, L-carnosine and their derivatives (e.g. anserine), carotenoids, carotenes (e.g. α-carotene, β-carotene, lycopene) and their derivatives, lipoic acid and its derivatives (e.g. dihydrolipoic acid), aurothioglucose, propylthiouracil and other thiols (e.g. thioredoxin, glutathione, cysteine, cysteine, cysteamine and their glycosyl, N-acetyl, methyl, ethyl, propyl, amyl, butyl and lauryl, palmitoyl, oleyl, γ-linoleyl, cholesteryl and glyceryl esters) and also their salts, dilauryl thiodipropionate, distearyl thiodipropionate, thiodipropionic acid and its derivatives (esters, ethers, peptides, lipids, nucleotides, nucleosides and salts) and also sulphoximine compounds (e.g. buthionine sulphoximine, homocysteine sulphoximine, methionine sulfone, penta-, hexa- and heptathionine sulphoximine) in very low tolerable doses (e.g. pmol to μmol/kg), furthermore (metal) chelators (e.g. α-hydroxy fatty acids, palmitic acids, lactoferrin), α-hydroxy acids (e.g. citric acid, lactic acid, maleic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbyl palmitate, Mg-ascorbyl phosphate, ascorbyl acetate), tocopherols and derivatives (e.g. vitamin E acetate) and also coniferyl benzoate of gum benzoin, rutic acid and its derivatives, ferulic acid and its derivatives, butylhydroxytoluene, butylhydroxyanisole, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenomethionine), stilbene and its derivatives (e.g. stilbene oxide, trans-stilbene oxide) and the derivatives suitable according to the invention (salts, esters, ethers, sugars, nucleotides, nucleosides, peptides and lipids) of the active compounds mentioned.

In one embodiment, the amount of the one or more antioxidants in the composition is from about 0.001% to about 30%, from about 0.05% to about 20%, from about 1% to about 10%, from about 0.1% to about 20%, from about 0.1% to about 19%, from about 0.1% to about 18%, from about 0.1% to about 17%, from about 0.1% to about 16%, from about 0.1% to about 15%, from about 0.1% to about 14%, from about 0.1% to about 13%, from about 0.1% to about 12%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 1% to about 20%, from about 1% to about 10%, from about 5% to about 20%, from about 10% to about 20%, from about 15% to about 20%, from about 1% to about 15%, from about 3% to about 10%, or from about 5% to about 7.5% by weight, based on the total weight of the composition.

In another embodiment, the antioxidant in the formulation is vitamin E, its derivatives or a mixture thereof, including acetate. In yet another embodiment, vitamin E, its derivatives or a mixture thereof is present in an amount from about 0.001% to about 30%, from about 0.001% to about 20%, from about 0.001% to about 10%, from about 0.05% to about 20%, from about 1% to about 10%, from about 0.1% to about 20%, from about 0.1% to about 19%, from about 0.1% to about 18%, from about 0.1% to about 17%, from about 0.1% to about 16%, from about 0.1% to about 15%, from about 0.1% to about 14%, from about 0.1% to about 13%, from about 0.1% to about 12%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 1% to about 20%, from about 1% to about 10%, from about 5% to about 20%, from about 10% to about 20%, from about 15% to about 20%, from about 1% to about 15%, from about 3% to about 10%, or from about 5% to about 7.5% by weight, based on the total weight of the formulation.

If the cosmetic composition within the meaning of the present invention is a solution or emulsion or dispersion, the following can be used as solvents: water or aqueous solutions; oils, such as triglycerides of capric or of caprylic acid, but preferably castor oil; fats, waxes and other natural and synthetic fatty materials, preferably esters of fatty acids with alcohols of low C number, e.g. with isopropanol, propylene glycol or glycerol, or esters of fatty alcohols with alkanoic acids of low C number or with fatty acids; alcohols, diols or polyols of low C number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In one specific embodiment, mixtures of the above-mentioned solvents are used. In the case of alcoholic solvents, water can be a further constituent.

The oil phase of the emulsions, oleogels or hydrodispersions or lipodispersions within the meaning of the present invention can advantageously be selected from the group consisting of the esters of saturated and/or unsaturated, branched and/or unbranched alkane carboxylic acids of a chain length of 3 to 30 C atoms and saturated and/or unsaturated, branched and/or unbranched alcohols of a chain length of 3 to 30 C atoms, from the group consisting of the esters of aromatic carboxylic acids and saturated and/or unsaturated, branched and/or unbranched alcohols of a chain length of 3 to 30 C atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and also synthetic, semisynthetic and natural mixtures of such esters, e.g. jojoba oil.

In one embodiment, the oil phase is selected from the group consisting of the branched and unbranched hydrocarbons and hydrocarbon waxes, the silicone oils, the dialkyl ethers, the group consisting of the saturated or unsaturated, branched, or unbranched alcohols, and also the fatty acid triglycerides, namely the triglycerol esters of saturated and/or unsaturated, branched and/or unbranched alkanecarboxylic acids of a chain length of 8 to 24, in particular 12-18, C atoms. The fatty acid triglycerides can, for example, be advantageously selected from the group consisting of the synthetic, semisynthetic, and natural oils, e.g., olive oil, sunflower oil, soya bean oil, ground nut oil, rape seed oil, almond oil, palm oil, coconut oil, palm kernel oil and the like.

### B. Anti-Inflammatory Compositions

In an aspect, IP-6 and/or inositol compositions of the present invention can be formulated with an anti-inflammatory agent in a cosmetic base or dental linament (periodontal disease) for topical application for local prevention of inflammation and/or tissue damage consequent to inflammation. A variety of steroidal and non-steroidal anti-inflammatory agents can be combined with IP-6 and/or inositol compounds.

In one embodiment, steroidal anti-inflammatory agents, including but are not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, flupreclnisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. In one specific embodiment, the steroidal anti-inflammatory for use in the present invention is hydrocortisone.

Specific non-steroidal anti-inflammatory agents useful in the composition of the present invention include, but are not limited to: piroxicam, isoxicam, tenoxicam, sudoxicam, CP-14,304, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, acemetacin, fentiazac, zomepirac, clidanac, oxepinac, felbinac, mefenamic, meclofenamic, flufenamic, niflumic, tolfenamic acids, ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, tiaprofenic, phenylbutazone, oxyphenbutazone, feprazone, azapropazone, and trimethazone, among others. Mixtures of these non-steroidal anti-inflammatory agents may also be employed, as well as the pharmaceutically-acceptable salts and esters of these agents. For example, etofenamate, a flufenamic acid derivative, is particularly useful for topical application. Of the nonsteroidal anti-inflammatory agents, ibuprofen, naproxen, flufenamic acid, mefenamic acid, meclofenamic acid, piroxicam and felbinac are preferred and ibuprofen, naproxen, and flufenamic acid are most preferred.

In yet another embodiment, so-called “natural” or “plant-based” anti-inflammatory agents are useful in the present invention. For example, candelilla wax, alpha bisabolol, aloe vera, Manjistha (extracted from plants in the genus Rubia, particularly Rubia cordifolia), and Guggul (extracted from plants in the genus Commiphora, particularly Commiphora mukul), may be used.

While aqueous solvents are generally preferred, the pharmaceutical/cosmetic compositions of the present invention are formulated as solutions, and they may include a pharmaceutically- or cosmetically-acceptable organic solvent. In one embodiment, the organic solvent is propylene glycol, polyethylene glycol (200-600), polypropylene glycol (425-2025), glycerol, 1,2,4-butanetriol, sorbitol esters, 1,2,6-hexanetriol, ethanol, butanediol, or a mixture thereof. In one specific embodiment, the solvent is isopropanol. In another embodiment, the organic solvent may be mixed with each other or with water.

In yet another embodiment, the composition of the present inventor contains an anti-inflammatory agent in an amount from about 0.01% to about 30%, from about 0.01% to about 20%, from about 0.01% to about 10%, from about 0.5% to about 20%, from about 1% to about 15%, from about 1% to about 10%, from about 1% to about 5%, from about 1% to about 4%, from about 1% to about 3%, from about 1% to about 2%, from about 0.1% to about 15%, from about 0.1% to about 14%, from about 0.1% to about 13%, from about 0.1% to about 12%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 1% to about 20%, from about 1% to about 10%, from about 5% to about 20%, from about 10% to about 20%, from about 15% to about 20%, from about 1% to about 15%, from about 3% to about 10%, or from about 5% to about 7.5% by weight, based on the total weight of the composition.

In one specific embodiment, the composition of the present inventor contains an anti-inflammatory agent in an amount from about 0.01% to about 5%, or from about 0.5% to about 2% by weight, based on the total weight of the composition.

In one embodiment, emollients include the following: hydrocarbon oils and waxes, including mineral oil, petrolatum, paraffin, ceresin, ozokerite, microcrystalline wax, polyethylene, and perhydrosqualene; silicone oils, such as dimethyl polysiloxanes, methyl phenyl polysiloxanes, water-soluble and alcohol-soluble silicone glycol copolymers; triglyceride esters, for example vegetable and animal fats and oils, including castor oil, safflower oil, cottonseed oil, corn oil, olive oil, cod liver oil, almond oil, avocado oil, palm oil, sesame oil, and soybean oil; acetoglyceride esters, such as acetylated monoglycerides; ethoxylated glycerides, such as ethoxylated glyceryl monostearate; alkyl esters of fatty acids having 10 to 20 carbon atoms, such as methyl, isopropyl, and butyl esters of fatty acids, hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate; alkenyl esters of fatty acids having 10 to 20 carbon atoms, including oleyl myristate, oleyl stearate, and oleyl oleate; fatty acids having 10 to 20 carbon atoms such as pelargonic, lauric, myristic, palmitic, stearic, isostearic, hydroxystearic, oleic, linoleic, ricinoleic, arachidic, behenic, and erucic acids; fatty alcohols having 10 to 20 carbon atoms such as lauryl, myristyl, cetyl, hexadecyl, stearyl, isostearyl, hydroxystearyl, oleyl, ricinoleyl, behenyl, and erucyl alcohols, as well as 2-octyl dodecanol; fatty alcohol ethers such as ethoxylated fatty alcohols of 10 to 20 carbon atoms, including the lauryl, cetyl, stearyl, isostearyl, oleyl, and cholesterol alcohols having attached thereto from 1 to 50 ethylene oxide groups or 1 to 50 propylene oxide groups; ether-esters such as fatty acid esters of ethoxylated fatty alcohols; lanolin and derivatives, such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; polyhydric alcohols and polyether derivatives such as propylene glycol, dipropylene glycol, polypropylene glycols 2,000 and 4,000, polyoxyethylene polyoxypropylene glycols, polyoxypropylene polyoxyethylene glycols, glycerol, sorbitol, ethoxylated sorbitol, hydroxypropyl sorbitol, polyethylene glycols 200-6,000, methoxy polyethylene glycols 350, 550, 750, 2,000 and 5,000, poly [ethylene oxide] homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C15-C18 vicinal glycol, and polyoxypropylene derivatives of trimethylolpropane; polyhydric alcohol esters, including ethylene glycol mono- and di-fatty acid esters, diethylene glycol mono- and di-fatty acid esters, polyethylene glycol (200-6,000) mono- and di-fatty acid esters, propylene glycol mono- and di-fatty acid esters, polypropylene glycol 2,000 monooleate, polypropylene glycol 2,000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters; wax esters such as beeswax, spermaceti, myristyl myristate, stearyl stearate; beeswax derivatives, e.g. polyoxyethylene sorbitol beeswax which are reaction products of beeswax with ethoxylated sorbitol of varying ethylene oxide content, forming a mixture of ether-esters; vegetable waxes including carnauba and candelilla waxes; phospholipids, such as lecithin and derivative; sterols, such as cholesterol and cholesterol fatty acid esters; and amides such as fatty acid amides, ethoxylated fatty acid amides, solid fatty acid alkanolamides.

In yet another embodiment, useful emollients which provide skin conditioning are glycerol, hexanetriol, butanetriol, lactic acid and its salts, urea, pyrrolidone carboxylic acid and its salts, amino acids, guanidine, digylcerol and triglycerol. Preferred skin conditioning agents are the propoxylated glycerol derivatives.

The compositions of the present invention may also be delivered by spray. Suitable propellants for cosmetic compositions within the meaning of the present invention, which can be sprayed from aerosol containers are the customary known easily volatile, liquefied propellants, for example hydrocarbons (propane, butane, isobutane), which can be employed on their own or as a mixture. Compressed air can also be advantageously used.

A person skilled in the art knows that there are non-toxic propellant gases which would be suitable for the present invention in the form of aerosol compositions.

### III. METHODS OF TREATMENT

Erythema is redness of skin caused by increased blood flow in the superficial capillaries usually resulting from skin injury, inflammation—non-specific, dermatitis, insect bite, infection, massage, waxing or tweezing of hairs, radiotherapy, etc.

Eczema is inflammation of the skin (dermatitis). It is characterized by itchiness and red coloration (erythema), sometimes with oozing and scarring. The affected area can be small, large, or even the entire body. The cause of eczema is unknown. However, environmental and genetic factors are presumed to be responsible. There is no known cure for eczema. The treatments are symptomatic—to reduce inflammation and relieve itching.

Leukoderma, also called vitiligo, is a disease that causes loss of pigmentation on the skin. The condition causes white patches to appear on the skin and is more distinguishable in people with dark skin. It is a long-term skin ailment that at times is incurable. With the help of proper medical diagnosis and a combination of various treatments, patients can manage the disease. All patients do not respond in the same way to the treatments due to different underlying causes; hence the results of the treatment will differ from one patient to another. Immune dysfunction, specifically autoimmunity is one of the pathogenetic factors for vitiligo. Thus, immune modulators such as IP-6 and inositol may be of help in this condition.

Rash is a skin area that is swollen, red, or irritated. Rashes on the skin can happen when the skin comes in contact with irritating substances like urushiol which is released from poison ivy, poison oak, or poison sumac. Rash can also form from touching oil-contaminated objects, such as gardening tools, clothes, or a pet's fur. Rash is an allergic reaction. Contact dermatitis is a form of rash.

Melasma is one of the most frequently acquired hyperpigmentation disorders, with a prevalence of around 1 to 5% in the general population, or as high as 30% in specific populations such as in Latin America. It usually presents in middle-aged women as asymptomatic brown patches on the face, with symmetrical disposition and irregular borders. The disease is influenced by factors such as sun exposure (including visible light), genetic background, and female sex hormones.

Initially considered a melanocyte dysfunction, Melasma's pathophysiology seems to be more complex, also involving keratinocytes, inflammatory cells, increased vascularization, abnormal elastic fibers, and basement membrane disruption, leading dermatologists to consider it as a photoaging skin disease. Despite the existence of effective therapies, this condition remains a challenge for the dermatologist as the clinical history of the disease usually involves inconsistent therapeutic results and almost constant relapses. The mainstay of melasma treatment remains prevention of exposure to sunlight and depigmenting topical agents. Oxidative damage has been considered to be a factor in the pathogenesis of melasma. Hence antioxidants may be useful in treatment of this condition.

Melanoma is the most serious type of skin cancer of the melanocytes that produce melanin. The risk of melanoma seems to be increasing in people under 40, especially women.

Carcinoma is a cancer that forms in epithelial tissue. Epithelial tissue lines most organs in the human body, including the skin. Basal cell carcinoma is a common skin cancer of the sun-exposed area of the face. Basal cell carcinomas are thought to appear from long-term exposure to ultraviolet (UV) radiation from sunlight.

Actinic keratosis is a precancerous skin condition that presents as rough, scaly patches or growths. Actinic keratosis can be caused by sun damage sustained over many years.

Psoriasis is a skin disease that causes red, itchy scaly patches, most commonly on the knees, elbows, trunk, and scalp. It is a common chronic disease that tends to go through cycles, flaring for a few weeks or months, then subsiding for a while or going into remission. Treatments are available only to manage symptoms.

Shingles is a skin disease that can be painful and is often caused by Herpes zoster.

Signs of sun damage can take years to visibly develop, for instance, after one year or more, five years or more, or one to two decades or more. Signs of sun damage become visible such as freckles, age spots, and wrinkles. Other conditions that can develop from sun exposure-related damage include precancerous skin growths, skin cancer, or both. The Sun prematurely ages the skin, known as photoaging. Damage the sun does to skin can be referred to by names, including photoaging, photodamage, solar damage, or sun damage. A common condition associated with premature aging of skin from sun exposure is wrinkles, which are visible lines and creases that form in the skin, and are commonly formed by the natural aging process. Treatments to improve the youthful appearance of skin include skin protection creams, cleansing milks, sunscreen lotions, suntan lotions, nourishing creams, moisturizers, etc.

In one embodiment, the composition of the present invention can be used to treat a variety of disease and conditions, including erythema, superficial (1st degree) thermal burns, eczema, psoriasis, and other skin ailments such as skin rashes, poison ivy, melanoma, carcinoma including basal cell carcinoma, actinic keratosis, ageing-related damages, sun exposure-related damages, red and/or irritated skin, leukoderma, shingles, appearance of wrinkles, and melasma.

In an embodiment, the composition of the present invention can be used to treat a variety of disease and conditions, including erythema, superficial (1st degree) thermal burns, eczema, psoriasis, and other skin ailments such as skin rashes, poison ivy, melanoma, carcinoma including basal cell carcinoma, actinic keratosis, ageing-related damages, sun exposure-related damages, red and/or irritated skin, leukoderma, shingles, appearance of wrinkles, and melasma in children of various ages.

In another embodiment, the composition of the present invention can be used to treat a variety of disease and conditions, and the appearance of said diseases and conditions, including erythema, superficial (1^{st} degree) thermal burns, eczema, psoriasis, and other skin ailments such as skin rashes, poison ivy, melanoma, carcinoma including basal cell carcinoma, actinic keratosis, ageing-related damages, sun exposure-related damages, red and irritated skin, leukoderma, shingles, appearance of wrinkles, and melasma in adults of various ages, including the elderly. The composition can also treat such conditions in patients in need thereof.

In another embodiment the invention provides a method of treatment of thermal burns of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of superficial thermal skin burn comprising topically applying approximately 0.1% to about 20% inositol and 0.1% to about 20% IP-6 compounds, by weight, in a suitable topical lotion or gel form. The lotion or gel is applied to the skin every two or three hours. In one specific embodiment, the composition comprises 4% by weight of Ca—Mg-IP-6 and inositol.

In one specific embodiment, the invention provides a method of treatment of thermal burns of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of thermal burns of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment is a method of treatment of thermal burns of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition additionally comprises 4% by weight of inositol. In one specific embodiment, the composition comprises IP-6 and inositol in a ratio from about 10:1 to about 1:10

In another embodiment the invention, provides a method of treating the appearance of thermal burns of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of superficial thermal skin burn comprising topically applying approximately 0.1% to about 20% inositol and 0.1% to about 20% IP-6 compounds, by weight, in a suitable topical lotion or gel form. The lotion or gel is applied to the skin every two or three hours. In one specific embodiment, the composition comprises 4% by weight of Ca—Mg-IP-6 and inositol.

In one specific embodiment, the invention provides a method of treating the appearance of thermal burns of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of thermal burns of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment is a method of treating the appearance of thermal burns of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition additionally comprises 4% by weight of inositol. In one specific embodiment, the composition comprises IP-6 and inositol in a ratio from about 10:1 to about 1:10

In another embodiment, the invention provides a method of treatment of eczema in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of eczema in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of eczema in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

In yet another embodiment, the invention provides a method of treatment of eczema in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition additionally comprises 4% by weight of inositol.

In another embodiment, the invention provides a method of treating the appearance of eczema in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of eczema in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of eczema in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

In yet another embodiment, the invention provides a method of treating the appearance of eczema in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition additionally comprises 4% by weight of inositol.

In another embodiment the invention provides a method of treatment of psoriasis in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of psoriasis in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of psoriasis in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

In yet another embodiment, the invention provides a method of treatment of psoriasis in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention provides a method of treating the appearance of psoriasis in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of psoriasis in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of psoriasis in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

In yet another embodiment, the invention provides a method of treating the appearance of psoriasis in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treatment of poison ivy of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of poison ivy of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of poison ivy of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treatment of poison ivy to an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treating the appearance of poison ivy of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of poison ivy of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of poison ivy of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treating the appearance of poison ivy to an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention provides a method of treatment melanoma in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of melanoma in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of melanoma in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and inositol.

In yet another embodiment, the invention provides a method of treatment of melanoma in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention provides a method of treating the appearance of melanoma in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of melanoma in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of melanoma in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and inositol.

In yet another embodiment, the invention provides a method of treating the appearance of melanoma in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treatment of carcinoma in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol. In yet another specific embodiment, the carcinoma is basal cell carcinoma.

In one specific embodiment, the invention provides a method of treatment of carcinoma of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6. In yet another specific embodiment, the carcinoma is basal cell carcinoma.

In another specific embodiment, the invention provides a method of treatment of carcinoma of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol. In yet another specific embodiment, the carcinoma is basal cell carcinoma.

Yet another embodiment provides a method of treatment of carcinoma of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol. In yet another specific embodiment, the carcinoma is basal cell carcinoma.

In another embodiment the invention, provides a method of treating the appearance of carcinoma in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol. In yet another specific embodiment, the carcinoma is basal cell carcinoma.

In one specific embodiment, the invention provides a method of treating the appearance of carcinoma of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6. In yet another specific embodiment, the carcinoma is basal cell carcinoma.

In another specific embodiment, the invention provides a method of treating the appearance of carcinoma of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol. In yet another specific embodiment, the carcinoma is basal cell carcinoma.

Yet another embodiment provides a method of treating the appearance of carcinoma of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol. In yet another specific embodiment, the carcinoma is basal cell carcinoma.

In another embodiment the invention, provides a method of treatment of actinic keratosis in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of actinic keratosis of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of actinic keratosis of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treatment of actinic keratosis of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treating the appearance of actinic keratosis in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of actinic keratosis of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of actinic keratosis of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treating the appearance of actinic keratosis of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treatment of sun exposure-related damage of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the sun exposure-related damages occur over a long period of time. In another specific embodiment, the long-term sun exposure-related damage occurs over one or two decades. In yet another specific embodiment, the sun exposure-related damage occurs after one year or more. In yet another specific embodiment, the composition comprises 4% by weight of Ca—Mg-IP-6 and inositol. In yet another specific embodiment, the composition comprises 4% by weight of Ca—Mg-IP-6 and 4% by weight of inositol.

In one specific embodiment, the invention provides a method of treatment of sun exposure-related damage of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6. In another specific embodiment, the sun exposure-related damage occur over a long period of time. In yet another specific embodiment, the sun exposure-related damage occur over one year or more, five years or more, or one or two decades or more. In yet another specific embodiment, the sun exposure-related damages occur after one year or more.

In another specific embodiment, the invention provides a method of treatment of sun exposure-related damage of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol. In another specific embodiment, the sun exposure-related damage occurs over a long period of time. In yet another specific embodiment, the sun exposure-related damage occurs over one or two decades or more. In yet another specific embodiment, the sun exposure-related damages occur after one year or more, or five years or more.

Yet another embodiment provides a method of treatment of sun exposure-related damage of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol. In yet another specific embodiment, the sun exposure-related damage occurs over a long period of time. In yet another specific embodiment, the sun exposure-related damage occurs over one or two decades. In yet another specific embodiment, the sun exposure-related damages occur after one year or more, or five years or more.

In another embodiment the invention, provides a method of treating the appearance of sun exposure-related damage of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the sun exposure-related damages occur over a long period of time. In another specific embodiment, the long-term sun exposure-related damage occurs over one or two decades. In yet another specific embodiment, the sun exposure-related damage occurs after one year or more. In yet another specific embodiment, the composition comprises 4% by weight of Ca—Mg-IP-6 and inositol. In yet another specific embodiment, the composition comprises 4% by weight of Ca—Mg-IP-6 and 4% by weight of inositol.

In one specific embodiment, the invention provides a method of treating the appearance of sun exposure-related damage of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6. In another specific embodiment, the sun exposure-related damage occur over a long period of time. In yet another specific embodiment, the long-term sun exposure-related damage occur over one year or more, five years or more, or one or two decades or more.

In another specific embodiment, the invention provides a method of treating the appearance of sun exposure-related damage of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol. In another specific embodiment, the sun exposure-related damage occurs over a long period of time. In yet another specific embodiment, the long-term sun exposure-related damage occurs over one or two decades or more. In yet another specific embodiment, the sun exposure-related damages occur after one year or more, or five years or more.

Yet another embodiment provides a method of treating the appearance of sun exposure-related damage of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In another specific embodiment, the composition comprises 4% by weight of inositol. In yet another specific embodiment, the sun exposure-related damage occurs over a long period of time. In another specific embodiment, the long-term sun exposure-related damage occurs over one or two decades. In yet another specific embodiment, the sun exposure-related damages occur after one year or more, or five years or more.

In another embodiment the invention, provides a method of treating the appearance of skin damage of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In yet another specific embodiment, the composition comprises 4% by weight of Ca—Mg-IP-6 and inositol. In yet another specific embodiment, the composition comprises 4% by weight of Ca—Mg-IP-6 and 4% by weight of inositol.

In one specific embodiment, the invention provides a method of treating the appearance of skin damage of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of skin damage of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treatment of treating the appearance of skin damage of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of improving the appearance of skin of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In yet another specific embodiment, the composition comprises 4% by weight of Ca—Mg-IP-6 and inositol. In yet another specific embodiment, the composition comprises 4% by weight of Ca—Mg-IP-6 and 4% by weight of inositol.

In one specific embodiment, the invention provides a method of improving the appearance of skin of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of improving the appearance of skin of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of improving the appearance of skin of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treatment of red and/or irritated skin of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of red and irritated skin of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of red and irritated skin of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treatment of red and irritated skin of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treating the appearance of red and/or irritated skin of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of red and irritated skin of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of red and irritated skin of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treating the appearance of red and irritated skin of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention provides a method of treatment of shingles in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of shingles in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of shingles in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and inositol.

In yet another embodiment, the invention provides a method of treatment of shingles in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention provides a method of treating the appearance of shingles in an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treating the appearance of shingles in an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of shingles in an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and inositol.

In yet another embodiment, the invention provides a method of treating the appearance of shingles in an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In another embodiment the invention, provides a method of treatment of the appearance of wrinkles of an individual in need thereof, comprising topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In one specific embodiment, the invention provides a method of treatment of the appearance of wrinkles of an individual in need thereof, comprising topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treatment of the appearance of wrinkles of an individual in need thereof, comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treatment of the appearance of wrinkles of an individual in need thereof, comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In one embodiment, the invention provides a method of moisturizing or improving the moisture content of skin comprising administering a composition comprising IP6 and inositol. Although not to be bound by theory, it is believed that the composition improve circulation to the skin, among other benefits. The method comprises topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In another embodiment, the invention provides a method of moisturizing or improving the moisture content of skin comprising administering a composition comprising IP6 and inositol. The composition may improve circulation to the skin. The method comprises topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of moisturizing or improving the moisture content of skin comprising administering a composition comprising IP6 and inositol comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of moisturizing or improving the moisture content of skin comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In one embodiment, the invention provides a method of treating the appearance of ageing diseases comprising administering a composition comprising IP6 and inositol. Although not to be bound by theory, it is believed that the composition improve circulation to the skin, among other benefits. The method comprises topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In another embodiment, the invention provides a method of treating the appearance of ageing diseases comprising administering a composition comprising IP6 and inositol. The composition may improve circulation to the skin. The method comprises topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of treating the appearance of ageing diseases comprising administering a composition comprising IP6 and inositol comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of treating the appearance of ageing diseases comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

In one embodiment, the invention provides a method of improving the youthful appearance of skin comprising administering a composition comprising IP6 and inositol. Although not to be bound by theory, it is believed that the composition improve circulation to the skin, among other benefits. The method comprises topically administering a therapeutically effective amount of a composition comprising IP-6 (its pharmaceutically acceptable salts, or its pharmaceutically acceptable derivatives, in any combination) or a combination of IP-6 and inositol (their pharmaceutically acceptable salts, or their pharmaceutically acceptable derivatives, in any combination). In one specific embodiment, the composition comprises 4% by weight of Ca—Mg-IP-6 and inositol. In another specific embodiment, the composition comprises 4% by weight of Ca—Mg-IP-6 and 4% by weight of inositol.

In another embodiment, the invention provides a method of improving the youthful appearance of skin comprising administering a composition comprising IP6 and inositol. The composition may improve circulation to the skin. The method comprises topically applying a therapeutically effective amount of a composition comprising IP-6, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6.

In another specific embodiment, the invention provides a method of improving the youthful appearance of skin comprising administering a composition comprising IP6 and inositol comprising topically applying a therapeutically effective amount of a composition comprising (i) IP-6, its pharmaceutically acceptable salts, or its pharmaceutically acceptable derivatives, in any combination, in an amount from about 0.1% to about 10% by weight of the composition; and (ii) inositol, its pharmaceutically acceptable salts, or its pharmaceutically acceptable derivatives, in any combination, in an amount from about 0.1% to about 10% by weight of the composition. In one specific embodiment, the composition comprises 4% by weight of Ca—Mg-IP-6 and 4% by weight of inositol.

Yet another embodiment provides a method of improving the youthful appearance of skin comprising topically applying a therapeutically effective amount of a composition comprising IP-6 and inositol, their pharmaceutically acceptable salts, or their pharmaceutically acceptable derivatives, in any combination. In one specific embodiment, the composition comprises IP-6 and inositol in a ratio from about 10:1 to about 1:10. In another specific embodiment, the composition comprises 4% by weight of Ca—Mg-IP-6 and inositol. In yet another specific embodiment, the composition comprises 4% by weight of inositol.

### IV. EXAMPLES

The following examples are included to demonstrate certain embodiments of the invention. Those of skill in the art should, however, in light of the present disclosure, appreciate that modifications can be made in the specific embodiments that are disclosed and still obtain a like or similar result without departing from the spirit and scope of the invention. Therefore all matter set forth is to be interpreted as illustrative and not in a limiting sense.

### Example 1—Topical Treatment of Eczema and Psoriasis Using Ca—Mg-IP-6 and Inositol

Several individuals with eczema (e.g., patient C.B.) and psoriasis (e.g., patient D.S.), were treated topically with a composition comprising 4% by weight of Ca—Mg-IP-6 and 4% by weight of inositol in a cream containing 1% colloidal oatmeal, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol. Patient C.B. was a 92-years old female who had eczema all her adult life that was uncontrolled. Surprisingly, following application of the cream, within about 6 hours the patient experienced marked relief of eczematous symptoms. Similar dramatic relief of symptoms of psoriasis were seen in others within 24-48 hours. Patient D.S. was a Caucasian woman in her mid to late thirties who had psoriasis. Similar dramatic relief of symptoms of psoriasis were seen in others within 24-48 hours.

### Example 2—Topical Treatment of Stage 1B Superficial Cutaneous Melanoma Using a Composition Comprising Ca—Mg-IP-6 and Inositol

One patient (C.S.) with Stage 1B superficial cutaneous melanoma was topically treated with a skin cream containing 4% by weight of Ca—Mg-IP-6 and inositol, thrice a day, in a 3.2:0.880 weight ratio. The cream also contained 1% colloidal oatmeal, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol. A marked improvement in the skin lesions were observed two weeks following the initiation of the treatment.

### Example 3—Treatment of Superficial Thermal Skin Burn Using a Composition Comprising Inositol and IP-6 Compounds

Patient R.T. (36-years old female) had repeatedly undergone superficial thermal burn of skin (1st degree) resulting in severe pain. Surprisingly, following application of a skin cream comprising 4% w/w Ca—Mg-IP-6 and inositol, in a 3.2:0.880 weight ratio, there was a near-instant pain relief, and resolution of skin redness and burn in 2-3 hours. The cream also contained 1% colloidal oatmeal, aqua (deionized water), glyceryl stearate, cetearyl olivate, cetyl alcohol, ethylhexylglycerin, glycerin, Helianthus annuus oil, inositol, phenoxyethanol, sorbitan olivate, xanthan gum and zenea propanediol.

Patient S T-U (65-years old female) had skin damage resulting from a history of sun exposure. Patient had been using generic moisturizers to treat skin regularly for years. Her skin was reported to be rough despite use of moisturizers. Patient applied cream formulation comprising IP6 and inositol at 4 wt-% and reported her skin felt “very soft” after the applications of said formulation.

### Example 4—Topical Treatment of Erythema Using a Composition Comprising IP-6 and Inositol

Erythema in the skin of a 5-years old child (S.P.) and in a 36-years old woman (R.T.) due to insect bite, abrasion, non-specific dermatitis, and poorly fitting garments were treated with a skin cream containing 4% by weight of Ca—Mg-IP-6 and inositol, in a 3.2:0.880 weight ratio. Surprisingly within an hour or two the redness completely disappeared. The results are unexpected. The cream also contained 1% colloidal oatmeal, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol.

While not wishing to be bound by theory, neither inositol nor IP-6 are known to decrease dilatation of capillaries though anti-angiogenesis action of IP-6 (Raina, et al., “Inositol Hexaphosphate Inhibits Tumor Growth, Vascularity, and Metabolism in TRAMP Mice: A Multiparametric Magnetic Resonance Study,” Cancer Prev Res. (2012), 6(1):40-50.). Raina, et al teaches that rats given 1-4% IP-6 in drinking water exhibited inhibited angiogenesis as evaluated by micro-vessel density (MVD) in experimental prostate cancer, measured by gadolinium uptake. Vucenik, et al. (“Anti-angiogenic activity of inositol hexaphosphate (IP-6),” Carcinogenesis. (2004), 11:2115-2123) used an in vitro model of capillary differentiation utilizing human umbilical vein endothelial cell (HUVEC) tube formation on reconstituted extracellular matrix. Following treatment with IP-6 there was a reduction in the number of capillaries after 24 hours. They also showed in an in vivo mouse model that IP-6 inhibited vascularity (number of blood vessel formation) 7 days following treatment. The research did not show any evidence that the capillaries or micro-vessels show any alteration in the luminal diameter, and certainly not within an hour or two, as was observed in this Example.

### Example 5—Topical Treatment of Rash Using a Composition Comprising Ca—Mg-IP-6 and Inositol

A 75-years old female had been exposed to poison ivy resulting in intense skin rashes on her exposed areas. The patient was treated topically with a composition comprising 4% by weight of Ca—Mg-IP-6 and inositol, in a 3.2:0.880 weight ratio, in a cream containing 1% colloidal oatmeal, deionized water, glyceryl stearate, cetearyl olivate, cetyl alcohol, ethylhexylglycerin, glycerin, Helianthus annuus oil, phenoxyethanol, sorbitan olivate, xanthan gum and zenea propanediol. The patient experienced amelioration of itching and marked improvement of the rashes in about 3 hours.

### Example 6—Topical Treatment of Poison Ivy Using a Composition Comprising Ca—Mg-IP-6 and Inositol

A 36-years old female of East Indian descent came in contact with poison ivy on her right thumb resulting in severe itching, redness, and swelling. Upon application of cream formulation comprising 200 mg of IP6 and inositol cream at 4-wt-%, within 20-30 minutes the itching and redness disappeared. Swelling persisted only for a few hours. A second application of said formulation resulted in amelioration of the swelling in approximately 2-3 hours.

### Example 7—Topical Treatment of Carcinoma Using a Composition Comprising Ca—Mg-IP-6 and Inositol

A 91-years old Caucasian woman was diagnosed with basal cell carcinoma of the skin on her face. The patient was treated topically with approximately 200-300 mg of the IP6 and Inositol cream, enough to cover the lesion, three times a day. The basal cell carcinoma of the skin cleared up following topical application of the cream.

### Example 8—Topical Treatment of Actinic Keratosis Using a Composition Comprising Ca—Mg-IP-6 and Inositol

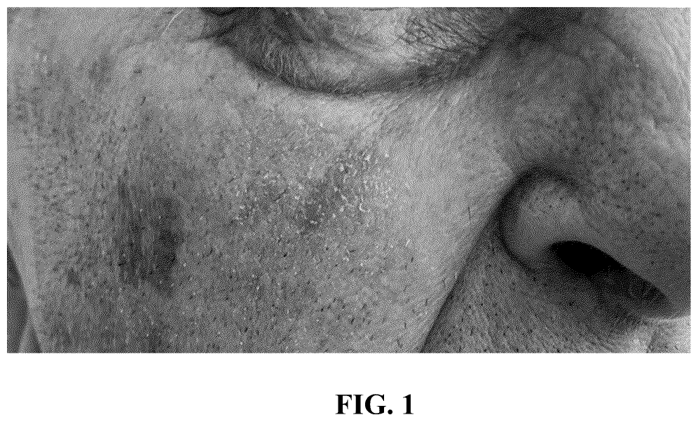

Patient J.V. (87-years old male) with skin cancer removed from his nose presented with precancerous (actinic keratosis) area under his right eye. Said actinic keratosis condition occurred due to long-term sun exposure. The patient was treated topically with a composition comprising IP6 and inositol at 4 wt-%. After a few days of treatment, the appearance of the redness and scaly white patches had improved significantly, and the skin was reported to be less dry or scaly to the touch. The results of the successful treatment are shown in FIG. 1.

Patient (80-years old man) presented with precancerous actinic keratosis. The patient was treated topically with a composition comprising 4 wt-% IP6 and inositol. After a few days the appearance of the redness and scaly white patches had improved significantly, and the skin was reported to be less dry or “scaly” to the touch.

The following publications are hereby incorporated by reference in their entireties:
1. U.S. Pat. No. 5,082,833, issued on Jan. 21, 1992
2. U.S. Patent Publication No. US 2007-0293458 A1
3. U.S. Pat. No. 7,517,868, issued on Apr. 14, 2009
4. U.S. Pat. No. 7,989,435, issued on Aug. 2, 2011
5. U.S. Pat. No. 7,009,067, issued 2006.
5. Riley, et al., “Cellular Internalisation of an Inositol Phosphate Visualised by Using Fluorescent InsP5,” ChemBioChem 2014, 15, 57-67.
6. Irvine, et al. “There is no ‘Conundrum’ of InsP6”; Open Biol. (2015) 5: 150181. http://dx.doi.org/10.1098/rsob.150181.
7. Grases, et al. “Study of the Absorption of Myo-Inositol Hexakisphosphate (InsP6) through the Skin,” Biol. Pharm. Bull. (2005), 28(4) 764-767.
8. Proietti, et al. “Inositol hexaphosphate (InsP6) as an Effective Topical Treatment for Patients Receiving Adjuvant Chemotherapy After Breast Surgery,” Eur Rev Med Pharmacol Sci, 2017, 21: 43-50.
9. Amabile, et al. “Effects of Inositol Hexaphosphate and Myo-Inositol Administration in Breast Cancer Patients during Adjuvant Chemotherapy,” J. Pers. Med. 2021, 11: 756.
10. Williams, et al. “Protective Effect of Inositol Hexaphosphate Against UVB Damage in HaCaT Cells and Skin Carcinogenesis in SKH1 Hairless Mice,” Comparative Medicine (2011), 61: 39-44.
11. Gupta, et al. “Suppression of DMBA-induced mouse skin tumor development by inositol hexaphosphate and its mode of action,” Nutr Cancer (2003), 46(1):66-72.
12. Ishikawa, et al. “Inhibition of Skin Cancer by IP6 in vivo: Initiation-Promotion Model,” Anticancer Res. (1999); 19(5A):3749-3752.
13. Piquero-Casals, et al. (“A Treatment Combination of Peels, Oral Antioxidants, and Topical Therapy for Refractory Melasma: A Report of 4 Cases,” Clinical, Cosmetic and Investigational Dermatology (2020), 13: 209-213.
14. Wawszczyk, et al., “Antiproliferative Effect of Inositol Hexaphosphate on Human Skin Melanoma Cells in vitro,” Acta Pol Pharm. (2015), 72(5):895-900.
15. Rizvi et al., “Inositol Hexaphosphate (IP-6) Inhibits Cellular Proliferation in Melanoma,” J Surg Res (2006), 133(1):3-6.
16. Khurana, et al., “Inositol Hexaphosphate Plus Inositol Induced Complete Remission in Stage IV Melanoma: A Case Report,” Melanoma Res. 2019; 29(3):322-324.
17. Wilson, et al. “A Novel Method for the Purification of Inositol Phosphates from Biological Samples Reveals that no Phytate is Present in Human Plasma or Urine,” Open Biol. (2015) 5: 150014.
18. Anthony L. L. Hunting (ed.), “A Formulary of Cosmetic Preparations (Vol. 2)—Creams, Lotions and Milks,” Nacelle Press (England, N.J. 1993).
19. Raina, et al., “Inositol Hexaphosphate Inhibits Tumor Growth, Vascularity, and Metabolism in TRAMP Mice: A Multiparametric Magnetic Resonance Study,” Cancer Prev Res. (2012), 6(1):40-50.
20 Vucenik, et al. “Anti-angiogenic activity of inositol hexaphosphate (IP-6),” Carcinogenesis. (2004), 11:2115-2123.
21. Martindale—The Extra Pharmacopoeia (Pharmaceutical Press, London 1993) and Martin (ed.), Remington's Pharmaceutical Sciences.

We claim:

1. A method for preventing or treating erythema of skin, skin rashes, poison ivy, superficial thermal burn, eczema, psoriasis, ageing-related skin damage, long-term sun exposure-related skin damage, red and irritated skin, non-specific dermatitis, melanoma, carcinoma, basal cell carcinoma, actinic keratosis, leukoderma, shingles, the appearance of wrinkles, and melasma, in a mammal in need thereof, wherein the method comprises topically applying a therapeutically effect amount of a composition comprising IP-6, its pharmaceutically acceptable salts, or its pharmaceutically acceptable derivatives, in an amount from about 0.1% to about 10% by weight of the composition.

2. The method of claim 1, wherein the composition further comprises inositol, its pharmaceutically acceptable salts, or its pharmaceutically acceptable derivatives, in an amount from about 0.1% to about 10% by weight of the composition.

3. The method of claim 2, wherein the composition further comprises at least one pharmaceutically acceptable excipient or carrier.

4. The method of any of claims 1-3, wherein the composition is a liquid, lotion, cream, gel, or ointment.

5. The method of claim 2, wherein the composition further comprises an antioxidant, a biocide, a chemotherapeutic, a nutritional supplement or nutraceutical, an analgesic, a sunblock, a moisturizer, or any combination thereof.

6. The method of claim 2, wherein the inositol and IP-6 are present in the forms of pharmaceutically acceptable salts, isomers, esters, derivatives, or any combination thereof.

7. A method for preventing or treating erythema of skin, skin rashes, poison ivy, superficial thermal burn, eczema, psoriasis, ageing-related skin damage, long-term sun exposure-related skin damage, red and irritated skin, non-specific dermatitis, melanoma, carcinoma, basal cell carcinoma, actinic keratosis, leukoderma, shingles, the appearance of wrinkles, and melasma, in a mammal in need thereof, wherein the method comprises topically applying a therapeutically effect amount of a composition comprising:
(i) IP-6, its pharmaceutically acceptable salts, or its pharmaceutically acceptable derivatives, in an amount from about 0.01% to about 10% by weight; and
(ii) inositol, its pharmaceutically acceptable salts, or its pharmaceutically acceptable derivatives, in an amount from about 0.01% to about 10% by weight.

8. The method of claim 7, wherein the composition comprises inositol and IP-6 or their pharmaceutically acceptable salts; in a molar ratio of from about 10:1 to about 1:10.

9. The method of claim 7, wherein the inositol and IP-6, their pharmaceutically acceptable salts, are present in a molar ratio from about 4:1 to about 1:4.

10. The method of any of claim 7, wherein the composition comprises:
(i) IP-6, its pharmaceutically acceptable salts, or its pharmaceutically acceptable derivatives, in an amount of from about 0.5% to about 5% by weight; and
(ii) inositol, its pharmaceutically acceptable salts, or its pharmaceutically acceptable derivatives, in an amount of from about 0.5% to about 5% by weight.

11. The method of claim 10, wherein the inositol and IP-6, their pharmaceutically acceptable salts, or their pharmaceutically acceptable derivatives, are present in a molar ratio of about 4:1 to about 1:4.

12. The method of claim 7, wherein the composition further comprises an antioxidant, a biocide, a chemotherapeutic, a nutritional supplement or nutraceutical, an analgesic, a sunblock, a moisturizer, or any combination thereof.

13. The method of any one of claims 8-12, wherein the inositol and IP-6 are present in the forms of pharmaceutically acceptable salts, isomers, esters, derivatives, or any combination thereof.

14. A composition comprising:
(i) IP-6, its pharmaceutically acceptable salts, or its pharmaceutically acceptable derivatives, in an amount from about 0.01% to about 10% by weight; and
(ii) inositol, its pharmaceutically acceptable salts, or its pharmaceutically acceptable derivatives, in an amount from about 0.01% to about 10% by weight.

15. A composition comprising: 4% by weight of Ca—Mg-IP-6, its pharmaceutically acceptable salts, or its pharmaceutically acceptable derivatives, and inositol, its pharmaceutically acceptable salts, or its pharmaceutically acceptable derivatives.

16. The composition of claim 15, wherein the inositol and IP-6 are present in a molar ratio from about 10:1 to about 1:10.

17. The composition of claim 16, wherein the inositol and IP-6, are present in a molar ratio from about 4:1 to about 1:4.

18. The method of claim 14-17, wherein the inositol and IP-6 are present in the forms of pharmaceutically acceptable salts, isomers, esters, derivatives, or any combination thereof.

19. A method for preventing or treating thermal burns of an individual in need thereof comprising topically applying a therapeutically effect amount of a composition comprising
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives.

20. A method for preventing or treating eczema in an individual in need thereof, wherein the method comprises topically applying a therapeutically effect amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives.

21. A method for preventing or treating psoriasis in an individual in need thereof, wherein the method comprises topically applying a therapeutically effect amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives.

22. A method for preventing or treating melanoma in an individual in need thereof, wherein the method comprises topically applying a therapeutically effect amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives, in any combination.

23. A method for preventing or treating erythema in an individual in need thereof, wherein the method comprises topically applying a therapeutically effect amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives, in any combination.

24. A method for preventing or treating rash in an individual in need thereof, wherein the method comprises topically applying a therapeutically effect amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives.

25. A method for preventing or treating basal cell carcinoma in an individual in need thereof, wherein the method comprises topically applying a therapeutically effect amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives, in any combination.

26. A method for preventing or treating shingles in an individual in need thereof, wherein the method comprises topically applying a therapeutically effect amount of a composition comprising: 4% by weight of Ca—Mg-IP-6, its pharmaceutically acceptable salts, or its pharmaceutically acceptable derivatives, and inositol, its pharmaceutically acceptable salts, or its pharmaceutically acceptable derivatives.

27. A method for improving the youthful appearance of skin in an individual in need thereof, comprising topically applying a therapeutically effective amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives;
wherein the method improves the youthful appearance of skin.

28. A method for improving the appearance of skin damage related to long-term sun exposure-related damage in an individual in need thereof, comprising topically applying a therapeutically effective amount of a composition comprising:
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives;
wherein the method improves the youthful appearance of skin.

29. The method of claim 28, wherein the sun exposure-related damage includes ageing-related damages, melanoma, carcinoma, basal cell carcinoma, actinic keratosis, wrinkles, or melisma.

30. The method of claim 28 where the long-term sun exposure-related damage occurs over one or two decades.

31. The method of claim 28 where the long-term sun exposure-related damage occurs after one year.

32. A method for moisturizing the skin comprising administering a composition comprising
(i) 4% by weight of Ca—Mg-IP-6, its pharmaceutically acceptable salts, or its pharmaceutically acceptable derivatives, and
(ii) inositol, its pharmaceutically acceptable salts, or its pharmaceutically acceptable derivatives.

33. A method for improving the moisture content of skin comprising topically administering a therapeutically effect amount of inositol and IP6, or pharmaceutically acceptable salts thereof.

34. A method for treating the appearance of damage related to long-term sun exposure in the skin of an individual, comprising topically administering a therapeutically effect amount of inositol and IP6, or a pharmaceutically acceptable salt thereof.

35. The method of claim 34, wherein the damage related to long-term sun exposure comprises melanoma, carcinoma, basal cell carcinoma, actinic keratosis, wrinkles, or melisma.

36. The method of claim 34, wherein the damage related to long-term sun exposure is caused by exposure to the sun for ten years or more.

37. A method for improving the youthful appearance of skin comprising topically administering a therapeutically effect amount of inositol and IP6, or a pharmaceutically acceptable salt thereof.

38. The method of claim 37 wherein the method improves the appearance of age-related damage, melanoma, carcinoma, basal cell carcinoma, actinic keratosis, wrinkles, or melisma.

39. A method for treating melanoma, carcinoma, basal cell carcinoma, actinic keratosis, wrinkles, or melisma comprising topically administering a therapeutically effect amount of inositol and IP6, or a pharmaceutically acceptable salt thereof.

40. A method for treating the appearance of melanoma, carcinoma, basal cell carcinoma, actinic keratosis, wrinkles, or melisma comprising topically administering a therapeutically effect amount of inositol and IP6, or a pharmaceutically acceptable salt thereof.

41. A composition comprising:
(i) 4% by weight of Ca—Mg-IP-6, its pharmaceutically acceptable salts, or its pharmaceutically acceptable derivatives, and
(ii) 4% by weight of inositol, its pharmaceutically acceptable salts, or its pharmaceutically acceptable derivatives.
